(19) United States
(12) Patent Application Publication
(10) Pub. No.: US 2021/0052689 A1
(43) Pub. Date: Feb. 25, 2021

(54) ANTI-MICROBIAL AMALGAMATE OF NON-TOXIC NATIVE COMPOSITION

(71) Applicant: Beneficial Biome Solutions, Inc, St. Louis, MO (US)

(72) Inventors: Douglas Steven Pernikoff, Glencoe, MO (US); Grant Lee Weber, St. Louis, MO (US)

(73) Assignee: Beneficial Biome Solutions, Inc, St. Louis, MO (US)

(21) Appl. No.: 17/001,440

(22) Filed: Aug. 24, 2020

Related U.S. Application Data
(60) Provisional application No. 62/891,261, filed on Aug. 23, 2019.

(51) Int. Cl. A61K 36/9068; A61K 47/44; A61K 36/02; A61K 36/45; A61K 36/23; A61K 36/53; A61K 36/48; A61K 36/07; A61K 35/63; A61K 36/534; A61K 36/752; A61K 36/54; A61K 35/64; A61K 36/537; A61K 36/185; A61P 31/10; A61P 31/04

(52) U.S. Cl. CPC A61K 36/9068; A61P 31/04; A61K 36/02; A61K 36/45; A61K 36/23; A61K 36/53; A61K 36/48; A61K 36/07; A61K 35/63; A61K 36/534; A61K 36/752; A61K 36/54; A61K 35/64; A61K 36/537; A61K 36/185; A61P 31/10; A61K 47/44

(57) ABSTRACT

An anti-microbial formulation of native ingredients for human and non-human vertebrate animal feeds, feed supplements/additives, or for direct doses to replace in whole or supplement traditional anti-microbial with side-effects such as allergies, microbial-resistance, and drug-interactions with non-toxic, side-effect free native anti-microbial for all vertebrates. The invention comprises of non-toxic, side-effect free native substances amalgamations having anti-microbial properties for food additives, food supplements, or direct doses. Furthermore, these native formulas can be use with or without other prescription or nonprescription anti-microbial medications. We describe the method of manufacture and also the process of use for this amalgamates.

### CROSS-REFERENCE TO RELATED APPLICATIONS

This application is a Non-Provisional application claiming priority of the Provisional U.S. Application No. 62/891,261 filed on Aug. 23, 2019, and Non-Provisional U.S. application Ser. No. 17/001,440. They are incorporated in entirety herein by references.

### STATEMENT REGARDING FEDERALLY SPONSORED RESEARCH OR DEVELOPMENT

Not applicable.

The present disclosure relates to a non-toxic, side-effect free, and native amalgamate with anti-microbial properties for food additives, food supplement(s), and direct dosing for vertebrates.

### BACKGROUND

A wide variety of pathogens causes infectious diseases in vertebrates and may be transmitted between and within species. An infected host develops a disease due to an invasive pathogen's reproductive multiplication and toxin production within an entire or partial host tissue, organs, and body. These pathogens or parasites can be single-cellular, prokaryotes, eukaryotes, or multicellular.

Often, the invading pathogen causes a disease in the host, but some invading microbial lay dormant in the host until they are transmitted to another species wherein, they cause disease. Hence, domesticated animals, farm animals, zoo animals, pets, wild animals, etc. can act as a carrier(s) to transmit a disease eventually to human hosts. Some animals form a source of food, and furthermore, consumption and handling of the infected foods, for example, dairy animals, poultry animals, meat-producing animals, etc., can cause transmission of the infectious disease easily into humans. A wild animal can also infect humans and non-wild animals because of hunting, attacks of humans and domesticated animals in populated areas adjacent to jungles, human and domesticated animal consumption of wild animals as food, shared ecosystem such as rivers, lakes, etc. Due to these reasons, the spread of infectious diseases across inter- and intra-species has become a significant challenge of the current civilization. It remains a considerable risk for our future. Hence, there is an enormous market need for a composition that can prevent the spread of infectious diseases and treat or cure them.

In response to the invading pathogen's multiplication and toxins, the host launches an immune response to eradicate the pathogens and combat the toxins. However, the immune response is not always effective and can cause symptoms such as inflammation, fever, etc. Medications (anti-microbial) assist the host's immune system in combating, eliminating, and eradicating the pathogens. These anti-microbial medications kill the pathogens or retard their growth and reproduction, or a combination of both. These synthetic medications are not side-effect free and can cause significant health problems after consumption, e.g., allergies, digestive disorders, drug-resistance, and several more. The invention described herein provides anti-microbial composition without toxic and side-effects at therapeutic or used doses.

### BRIEF DESCRIPTION OF THE INVENTION

This application relates to a unique amalgamation of native compositions having anti-microbial activities, for example, anti-pathogens, anti-mites, and anti-parasites activities. This composition may be a food additive or a food supplement for a variety of vertebrates such as, but not limited to, humans, farm animals, domesticated animals, pets, zoo animals, wild animals, etc. This unique and unpredictable combination of native ingredients having anti-microbial activity may prevent, treat, complement traditional treatments, cure, or any combination thereof for several infectious diseases. It may prevent the spread and transmission of several infectious diseases. Human contact with an animal may transmit the disease from humans to animals or vice versa. In contrast, direct or indirect physical contact between humans may spread these diseases from one human to another. Similar contact between humans and non-human vertebrates or between non-human vertebrates may spread the disease amongst humans and non-human vertebrates, respectively.

The anti-microbial activity of this invention may either kill, retard growth, or a combination of both. The microbe may be either unicellular or multicellular pathogen causing disease in any vertebrate. The vertebrate may serve a pathogenic host, non-pathogen host, as a carrier who transmits the pathogen to other hosts, or any combination thereof. This invention may, therefore, prevent, treat, complement traditional treatments of or cure infections in one, two three, four, or more vertebrates.

In one embodiment of this invention, the composition may prevent the spread of disease within a species (intra-species), between two or more species (inter-species), or a combination of both concomitantly and/or at different times.

In an embodiment of this invention, the composition may be used in conjunction with or without other compositions, which may be another anti-microbial composition. Another composition may or may not be prescribed by a health-care provider.

The composition of this invention may be non-toxic and side-effect free. All known anti-microbial therapeutics have various side effects, which may even be serious; a few side-effects are allergies, inflammation, drug-resistance, etc. However, an embodiment of this invention may not have any known adverse effects for any hosts at the therapeutic or recommended amounts used.

The ingredients of this invention may be natively occurring and may or may not be organic, GRAS certified, from certified grass-fed animals, or any combination thereof.

The composition of this invention may contain plant powder, plant oils, plant extracts, plant solids, plant liquids, etc. Another embodiment of this invention may use animal or insect proteins, fats, vitamins, fiber, etc. in the form of powder, oil, extract, aerosol, solids, liquids, etc.

The composition of this invention may contain inert or active thickeners from plants, microbes, and animals. The inert thickeners may be human-made or non-man-made.

The composition of this invention may further contain dilutants. The dilutants may be from plant or animal, for example, plant oil, animal oils, plant extract, animal extracts, plant liquid, animal liquids, fluids, or solids, water, native or human-made alcohols, aerosols, organic or non-organic solvents, etc.

The composition of this invention may have a variety of physical, for example, oil, emulsion, hydrogel, hydrogel admixture, pressurize or non-pressurized spray, seed cover, seed coat, paste, capsule, patches, pellets, oral-deliverables, injections, anal-deliverables, inhalable, syringe filling, mash, unmixed, mixed, blendable, unblendable, miscible, immiscible, or any combination thereof.

The user may consume this composition in 1, 2, 3, 4, 5, 6, 7, 8, 9, 10, 11, 12, 15, 16, 17, 18, 19, 20, 21, 22, 23, 24, 25, 26, 27, 28, 29, 30-35, 35-40, 40-45, 45-50, or more doses spanning over a few minutes, hours, days (1, 2, 3, 4, 5, 6, 7, 8, 9, 10, 11, 12, 13, 14, 15, 16, 17, 18, 19, 20-25, 25-30, 30-35, 35-40, 40-45, 45-50, 50-55, 55-60, or more day) weeks (1, 2, 3, 4, 5, 6, 7, 8, 9, 10, 11, 12, 13, 14, 15, 16, 17, 18, 19, 20, 21, 22, 23, or more weeks), months (1, 2, 3, 4, 5, 6, 7, 8, 9, 10, 11, 12, 13, 14, 15, 16, 17, 18, 19, 20 or more months), or upto 1, 2, 3, 4, 5, or more years.

The composition of this invention may prevent, treat, or cure a variety of microbial infections in any or all vertebrates.

### BRIEF DESCRIPTION OF FIGURES

FIG. 1: Composition A/B/C (1:100× dilution) inhibition of Salmonella and E. coli:

FIG. 2: Composition A/B/C (1:100× Dilution) Inhibition of Campylobacter jejuni.

FIG. 3. Composition A/B/C (1:100× Dilution) Inhibition of Candida albicans.

FIG. 4. Composition A/B/C Inhibition of Aspergillus fumigatus.

FIG. 5. Composition A/B/C Inhibition of (Anti-Microbial Resistance) AMR Salmonella.

In an embodiment of this invention, the composition may be manufactured in several forms such as, but not limited to, oils, mixture, amalgamate, solutions, patches, pellets, pills, oral-deliverables, injections, anal-deliverables, inhalable, emulsion, hydrogel, hydrogel admixture, pressurize or non-pressurized spray, seed cover, seed coat, paste, capsule, syringe filling, mash, unmixed, mixed, blendable, unblendable, aerosol, miscible, or immiscible.

### DETAILED DESCRIPTION OF THE INVENTION

The preceding summary, as well as the following detailed description of certain embodiments, will be better understood when read in conjunction with the appended figure of experimental data and results. As used herein, an element or step recited in the singular and proceeded with the word “a” or “an” should be understood as not excluding the plural of said elements or steps, unless such exclusion is explicitly stated. Furthermore, references to “one embodiment” or “an embodiment” are not intended to be interpreted as excluding the existence of additional embodiments that also incorporate the recited features. Moreover, unless explicitly stated to the contrary, embodiments “comprising” or “having” an element, or a plurality of elements having a particular property may include additional such elements not having that property. When a definition is provided herein, it supersedes any other meaning or definition.

As used herein, all percentages are expressed in weight by volume, unless otherwise stated.

As used herein, the term “amalgamate” means a mixture or combination of native ingredients of this invention.

As used herein, the term “microbial” means a microscopic organism that may be either single cellular, for example, but not limited to, virus, bacteria, fungi, yeasts, micro-algae, etc., or a multicellular, such as but not limited, ectoparasites, nematodes, ticks, lice, mites, worms, etc. or any combination thereof.

As used herein, the term “anti-microbial” means a composition that has properties of antimicrobial-cidal (killing the microbial), antimicrobial-static (slowing microbial growth), or any combination of both.

As used herein, the term “microbial transmission” means microbial transmission from any non-human vertebrate to humans, humans to any non-human vertebrates, between humans, and the microbes may transmit the pathogen between different two or more species or the same species.

As used herein, the term “vertebrate” means an organism that has a vertebra or a back-bone.

As used herein, the term “dilutant” means any material such as liquid, solid, or aerosol that may dilute a mixture, amalgamate, or a composition.

As used herein, the term “inert” means a substance that has no anti-microbial activity.

As used herein, the term “thickener” means a substance that increases the density of a mixture, amalgamate, or a composition.

As used herein, the term “inter-species” means between species.

As used herein, the term “intra-species” means within the same species.

As used herein, the term “native” means non-synthetic and non-human-made substances found in the ecosystem.

As used herein, the term “side-effect” means that any effect caused by the composition other than the intended, anti-microbial herein, effect, which is harmful at recommended doses or amounts of the composition and under normal recommended conditions of use. Thus, side-effect-free means without or free of the said effect(s) in any vertebrate.

As used herein, the term “toxic” means that the composition is harmful when ingested and/or comes in physical contact by any body part of any vertebrate. The harmful effects maybe, but are not limited to, burning sensation, confusion, drowsiness, fainting fits, loss of consciousness, coma, tissue destruction, vomiting, indigestion, stomach pain, skin-bruising, irritation of skin, eyes, and other parts of the body, blurry vision, coughing, nausea, shortness of breath, watery eyes, pain in the chest, throat, nose, eye irritation, wheezing, pneumonia, fluid in the lungs, irritation, inflammation, rash, etc. Thus, non-toxic means without such toxic effects in any vertebrates.

As used herein, the term “organic” means without pesticides, chemical fertilizers, synthetic antibiotics, and non-GMO and certified organic by the U. S. Department of Agriculture (USDA).

Antibiotics were introduced into feeds of animals as early as the 1950s. The purpose was to decrease the incidence of disease and death and improve growth rates and overall production performance. Despite the success in this policy for so many decades, today, there is a global threat of microbial organisms, particularly bacteria, viruses, fungi, and other anti-microbes, which have become more frequently and more quickly resistant to these same antibiotics that previously provided rescue from these microbes. The global community now faces daily threats to human and animal health challenged. Vertebrate infectious diseases caused by several microbes present directly or indirectly in vertebrate (including humans) living spaces and the consumed foods. Humans (and other vertebrates) handle food during chain processes and ingest foods microbial exposed microbial. These microbial are a variety of old and newly developed pathogens. Thus, humans (and other vertebrates) are facing the challenges confronting a world threatened by highly pathogenic, and even antibiotic-resistant microbial agents, encouraged by inappropriate human management practices, by climate change and due to fast-growing, concentrated human populations.

The composition includes all GRAS ingredients certified as safe and non-toxic, plant-derived essential oils, that work in concert to improve the intestinal microbiome in animals and man, without the untoward effects that historically used antibiotic feed supplements directly, or indirectly threaten their health and survivability

In an embodiment of the invention, a composition provides an anti-microbial activity. The composition contains a mixture of native substances, for example, herbal plants, krill, micro-algae, cranberry seed, chia, black cumin, carob, oregano, coriander, ginger, fenugreek, cinnamon, trans-cinnamaldehyde, cardamom, or any combination thereof. These plant products may be in powder, flour, oil, extract, solid, liquid, resin, diffusable, aerosol, vapor form, or any combination thereof.

Another embodiment of the invention further may contain other substances such as, but not limited to, Agaricus mushroom, moringa, kelp, insects, insect-parts, rosemary, mustard, fenugreek, thyme, turmeric, others, or any combination thereof. These substances may be in powder, flour, oil, extract, solid, liquid, resin, diffusable, aerosol, vapor form, or any combination thereof.

In another embodiment of the invention, the composition may have nutritional value, for example, may contain insect protein, insect fat, insect fiber, insect antioxidants such as, but not limited to, cricket, blackfly larvae, roaches, mopane worm, witchety grub, queen ants, others, or any combination thereof. Insects contain a complete protein with all nine essential amino acids, vitamins, minerals, for example, cricket contains several vitamins such as, but not limited to, vitamin A, B, and riboflavin, and they offer over triple the amount of B₁₂ as compared to salmon. They also contain five times more magnesium and iron as compared to beef and more calcium than milk. Crickets, grasshoppers, and silkworms have over three times the antioxidants as oranges. Also, insect fiber, namely chitin, is a prebiotic fiber and a great source of nutrients for probiotic gut bacteria.

An embodiment of the invention may further contain flavors such as, but not limited to, mint, peppermint, spearmint, menthol, basil, clove, cilantro, fennel, eucalyptus, garlic, lavender, lemon, lemon-grass, citrus, orange, rose, sage, lemon oil, etc., or any combination thereof. These may be powder, flour, oil, extract, solid, liquid, resin, diffusable, aerosol, vapor forms, or any combination thereof.

An embodiment of any of the composition further contains at least one or more dilutant such as, but not limited to, coconut oil, peanut oil, soybean oil, corn oil, canola oil, cottonseed oil, olive oil, palm oil, rapeseed oil, safflower oil, sesame oil, pumpkin oil, almond oil, grape seed oil, avocado oil, flax-seed oil, ghee, emu oil, alcohols, sunflower, or any combination thereof. These dilutant oils may be mechanically extracted, chemically extracted, or a combination of both, and maybe from a plant or an animal source. The dilutant may be liquid, solid, vapor, an aerosol, or any combination thereof.

Another embodiment of the composition further contains at least one or more thickeners such as, but not limited to, fumed silica, natural gum, starch, gelatin, agar, arrowroot, pectin, psyllium, sago, dextrin, cellulose, tapioca, tapioca starch, Xanthum gum, Kuzu/kudzu, Polyethylene glycol (PEG), waxes, etc., or any combination thereof.

Another embodiment of the composition further contains at least one or more solvents such as, but not limited to, isopropyl alcohol, ethanol, organic solvents, inorganic solvents, polar solvents, non-polar solvents, water, oils, alcohols, etc., or any combination thereof.

In one embodiment of the invention, the composition may be a paste, soft paste, mash, liquid, solid, semi-solid, gas, aerosol-like consistency, or any combination thereof.

The composition of this invention may be manufactured in several physical forms such as, but not limited to, emulsion, hydrogel, hydrogel admixture, solutions, patches, pellets, pills, oral-deliverables, injections, anal-deliverables, inhalable, pressurize or non-pressurized spray, top dressing, top coating, seed cover, seed coat, paste, capsule, syringe filling, unmixed, unblendable, blended, miscible, or immiscible, mash covering, commercial food covering, coating, prepared food additive, prepared food covering, prepared food coating, etc., or any combination thereof.

Another embodiment of this invention may be a cleaning agent with anti-microbial activity, such as but not limited to, an antiseptic, pressurize or non-pressurized spray, wash, liquid, or gel clearer, etc., or any combination thereof. An embodiment may provide hygiene and hygiene management control through the entire life cycle of any animal.

Another embodiment of this invention may be a cleaning agent with anti-mites activity, such as but not limited to, an antiseptic, pressurize or non-pressurized spray, wash, liquid or gel clearer, etc., or any combination thereof. An embodiment provides hygiene and management control through the entire life cycle of any animal.

Another embodiment of the composition may contain an effective amount of oregano oil, extract, powder, or juice 15%-40% by weight/volume, 20%-40% by weight/volume, 20%-35% by weight/volume, 23%-31% by weight/volume, 24%-30.5% by weight/volume, 23%-30% by weight/volume, 25%-27% by weight/volume, 15%-30% by weight/volume, 18%-25% by weight/volume, or any combination thereof.

Another embodiment of the composition may contains an effective amount of coconut powder, oil, extract, or juice 10%-40% by weight/volume, 15%-30% by weight/volume, 20%-35% by weight/volume, 18%-30% by weight/volume, 20%-25% by weight/volume, 21%-27% by weight/volume, or 21%-24% by weight/volume, or any combination thereof.

Another embodiment of the composition may contain an effective amount of chia flour, oil, extract, or juice 5%-30% by weight/volume, 10%-25% by weight/volume, 12%-22% by weight/volume, 13%-20% by weight/volume, 15%-20% by weight/volume, 16%-19% by weight/volume, or 16%-17% by weight/volume, or any combination thereof.

Another embodiment of the composition may contain effective amount of krill powder, oil, extract, or juice 5%-30% by weight/volume, 10%-25% by weight/volume, 12%-22% by weight/volume, 13%-20% by weight/volume, 15%-20% by weight/volume, 16%-19% by weight/volume, or 16%-17% by weight/volume, or any combination thereof.

Another embodiment of the composition may contain effective amount of micro-algae powder, oil, extract, or juice 5%-30% by weight/volume, 10%-25% by weight/volume, 12%-22% by weight/volume, 13%-20% by weight/volume, 15%-20% by weight/volume, 16%-19% by weight/volume, or 16%-17% by weight/volume, or any combination thereof.

Another embodiment of the composition may contain an effective amount of fumed silica 0.5%45% by weight/volume, 1%-12% by weight/volume, 1%40% by weight/volume, 3%-10% by weight/volume, 3%-9% by weight/volume, 4%-8% by weight/volume, or 5%-7% by weight/volume, or any combination thereof.

Another embodiment of the composition may contain an effective amount of cranberry seed powder, oil, extract, or juice 0.1%-10% by weight/volume, 0.5%-10% by weight/volume, 1%-9% by weight/volume, 1.5%-8% by weight/volume, 2.5%-7% by weight/volume, 2.5%-8% by weight/volume, 3%-7% by weight/volume, 3%-5% by weight/volume, or 3%-4% by weight/volume, or any combination thereof.

Another embodiment of the composition may contain an effective amount of coriander powder, extract, juice, or oil 0.1%-10% by weight/volume, 0.5%-9% by weight/volume, 1%-8% by weight/volume, 1.9%-7% by weight/volume, 2%-6% by weight/volume, 2%-5% by weight/volume, 2.5%-6% by weight/volume, 2.5%-5% by weight/volume, 2.5%-4 by weight/volume, or 2.5%-3.5% by weight/volume, or any combination thereof.

Another embodiment of the composition may contain an effective amount of ginger powder, oil, extract, or juice 0.1%-5% by weight/volume, 0.25%-5% by weight/volume, 0.5%-4% by weight/volume, 0.6%-3% by weight/volume, 0.6%-2% by weight/volume, 0.6%-1.5% by weight/volume, 0.6%-1% by weight/volume, or 0.5%-1% by weight/volume, or any combination thereof.

Another embodiment of the composition may contain an effective amount of black cumin powder, oil, extract, or juice 0.05%-5% by weight/volume, 0.1%-5% by weight/volume, 0.15%-4% by weight/volume, 0.2%-3% by weight/volume, 0.2%-2% by weight/volume, 0.2%-1.5% by weight/volume, 0.3%-1% by weight/volume, or 0.4%-1% by weight/volume, or any combination thereof.

Another embodiment of the composition may contain an effective amount of carob powder, oil, extract, or juice 0.1%-5% by weight/volume, 0.25%-5% by weight/volume, 0.5%-4% by weight/volume, 0.5%-3% by weight/volume, 0.5%-2% by weight/volume, 0.5%-1.5% by weight/volume, 0.5%-1% by weight/volume, and/or 0.6%-0.9% by weight/volume, or any combination thereof.

Another embodiment of the composition may contain an effective amount of fenugreek powder, oil, extract, or juice 0.01%-5% by weight/volume, 0.025%-5% by weight/volume, 0.1%-4% by weight/volume, 0.1%-3% by weight/volume, 0.1%-2% by weight/volume, 0.1%-1.5% by weight/volume, 0.1%-1% by weight/volume, and/or 0.2%-0.9% by weight/volume, or any combination thereof.

Another embodiment of the composition may contain an effective amount of agaricus mushroom powder, oil, extract, or juice 0.01%-5% by weight/volume, 0.025%-5% by weight/volume, 0.1%-4% by weight/volume, 0.1%-3% by weight/volume, 0.1%-2% by weight/volume, 0.1%-1.5% by weight/volume, 0.1%-1% by weight/volume, and/or 0.2%-0.9% by weight/volume, or any combination thereof.

Another embodiment of the composition may contain an effective amount of cardamom powder, oil, extract, or juice 0.01%-5% by weight/volume, 0.025%-5% by weight/volume, 0.1%-4% by weight/volume, 0.1%-3% by weight/volume, 0.1%-2% by weight/volume, 0.1%-1.5% by weight/volume, 0.1%-1% by weight/volume, and/or 0.2%-0.9% by weight/volume, or any combination thereof.

Another embodiment of the composition may contain an effective amount of oregano oil 11.0-24%, wt/wt, e.g., 11.0, 11.5, 11.6, 11.7, 11.8, 11.9, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.1, 23.2, 23.3, 23.4, 23.5, 23.6, 23.7, 23.8, 23.9, 24% wt/wt or any combination thereof. These are in % wt/wt.

Another embodiment of the composition may contain an effective amount of coconut oil 19-24.5%, wt/wt, e.g., 19, 19.5, 19.6, 10.7, 19.8, 19.8, 19.9, 20, 20.1. 20.2, 20.3, 20.4, 20.5, 21, 21.5, 22, 22.5, 23, 23.1, 23.2, 23.3, 23.4, 23.4, 23.5, 23.6, 23.7, 23.8, 23.9, 24, 24.1, 24.2, 24.3, 24.4, 24.5 or an combination thereof. These are in % wt/wt.

Another embodiment of the composition may contain an effective amount of elantria DHA 400 algae 15.-20% wt/wt, e.g., 15.1, 15.2, 15.3, 15.4, 15.5, 15.6, 15.7, 15.8, 15.9, 16, 16.1, 16.2, 16.3, 16.5, 16.6, 16.7, 16.8, 16.9, 17, 17.5, 17.6, 17.7, 17.8, 17.9, 18, 18.1, 18.2, 18.3, 18.4, 18.5, 18.6, 18.7, 18.8, 189.9, 19, 19.5, 20, or any combination thereof. These are in % wt/wt.

Another embodiment of the composition may contain an effective amount of MCT oil 21-26.5%, wt/wt, e.g., 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.5, 24, 24.5, 24.6, 24.7, 24.8, 24.9, 25, 25.1, 25.2, 25.3, 25.4, 25.5, 25.6, 25.7, 25.8, 25.9, 26, 26.5, or any combination thereof. These are in % wt/wt.

Another embodiment of the composition may contain an effective amount of cranberry see oil 2.5-4.0% wt/wt, e.g., 2.5, 2.6, 2.7, 2.8, 2.9, 3, 3.1, 3.2, 3.3, 3.4, 3.5, 3.6, 3.7, 3.8, 3.9, 4, or any combination thereof. These are in % wt/wt.

Another embodiment of the composition may contain an effective amount of coriander oil 2.-4% wt/wt, e.g., 2, 2.1, 2.2, 2.3, 2.4, 2.5, 2.6, 2.7, 2.8, 2.9, 3, 3.1, 3.2, 3.4, 3.5, 3.6, 3.7, 3.8, 3.9, 4, or any combination thereof. These are in % wt/wt.

Another embodiment of the composition may contain an effective amount of ginger oil 0.4-1.5% wt/wt, e.g., 0.4, 0.5, 0.6, 0.7, 0.8, 0.9, 1, 1.1, 1.2, 1.3, 1.4, 1.5, or any combination thereof. These are in % wt/wt.

Another embodiment of the composition may contain an effective amount of black cumin oil 0. 0.2-1.5% wt/wt, e.g., 0.2, 0.3, 0.4, 0.5, 0.6, 0.7, 0.8, 0.9, 1, 1.1, 1.2, 1.3, 1.4, 1.5, or any combination thereof. These are in % wt/wt.

Another embodiment of the composition may contain an effective amount of carob oil 0.2-1.5% wt/wt, e.g., 0.2, 0.3, 0.4, 0.5, 0.6, 0.7, 0.8, 0.9, 1, 1.1, 1.2, 1.3, 1.4, 1.5, or any combination thereof. These are in % wt/wt.

Another embodiment of the composition may contain an effective amount of fenugreek oil 0.2-1.5% wt/wt, e.g., 0.2. 0.3. 0.4, 0.5, 0.6, 0.7, 0.8, 0.9, 1, 1.1, 1.2, 1.3, 1.4, 1.5, or any combination thereof. These are in % wt/wt.

Another embodiment of the composition may contain an effective amount of agaricus oil 0.2-1.5% wt/wt, e.g., 0.2. 0.3, 0.4, 0.5, 0.6, 0.7, 0.8, 0.9, 1, 1.1, 1.2, 1.3, 1.4, 1.5, or any combination thereof. These are in % wt/wt.

Another embodiment of the composition may contain an effective amount of cinnamon oil 0.4-1.5% wt/wt, e.g., 0.4, 0.5, 0.6, 0.7, 0.8, 0.9, 1, 1.1, 1.2, 1.3, 1.4, 1.5, or any combination thereof. These are in % wt/wt.

Another embodiment of the composition may contain an effective amount of lemon oil 8.4-10.5% wt/wt, e.g., 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, or any combination thereof. These are in % wt/wt.

A user (host) may consume several forms and number of doses ranging from one to few to many spanning over a few minutes, hours, days, weeks, months, or years. The user may consume this composition in 1, 2, 3, 4, 5, 6, 7, 8, 9, 10, 11, 12, 15, 16, 17, 18, 19, 20, 21, 22, 23, 24, 25, 26, 27, 28, 29, 30-35, 35-40, 40-45, 45-50, more doses, or any combination thereof, and spanning over a few minutes, hours, days, for example, 1, 2, 3, 4, 5, 6, 7, 8, 9, 10, 11, 12, 13, 14, 15, 16, 17, 18, 19, 20-25, 25-30, 30-35, 35-40, 40-45, 45-50, 50-55, 55-60, or more day or any combination thereof; 1, 2, 3, 4, 5, 6, 7, 8, 9, 10, 11, 12, 13, 14, 15, 16, 17, 18, 19, 20, 21, 22, 23, or more weeks or any combination thereof; 1, 2, 3, 4, 5, 6, 7, 8, 9, 10, 11, 12, 13, 14, 15, 16, 17, 18, 19, 20, or more months or any combination thereof; or upto 1, 2, 3, 4, 5, more years or any combination thereof.

In one embodiment of the invention, the composition is diluted before dosing; the dilution is done before or after adding or mixing with prepared food or feed. The composition may be administrated via one, two, three, four, five, or more of several possible routes including, but not limited to, oral, transdermal, sublingual, anal, inhalable, injectables, etc., or any combination thereof. For example, chicks may be given a daily dose at top-dressing as 2%-4% dilution of the composition in commercial feed in the amount ranging from 6 mg to 17 mg per chick per day, e.g., 6 mg, 7 mg, 8 mg, 9 mg, 10 mg, 1 lmg, 12 mg, 13 mg, 14 mg, 15 mg, 16, 17 mg, or more, or any combination thereof. This dosing pattern applies to any small size vertebrate.

In one embodiment of the invention, the composition may be given at a daily dose at top-dressing as 2%-4% dilution of the composition in commercial feed in the amount ranging from pig dose is 0.5-3.0 gm per kg of feed, daily, for example, 0.5 mg, 1.0, 1.25, 1.5, 1.75, 2.0, 2.25, 2.5, 2.75, 3 gms or any combination thereof. This dosing pattern applies to any vertebrates of large size vertebrates, e.g., cows, horses, etc.

In one embodiment of the invention, the composition may be given at a daily dose of 35-50 gm per day, for example, 30, 34, 35, 36, 37, 38, 39, 40, 41, 42, 43, 44, 45, 46, 47, 48, 49, 50, 51, 52, 53, 54, 55, 56 gms or any combination thereof per day, for humans. This dosing pattern applies to human size vertebrates.

An embodiment of the composition may be used prophylactically to prevent infections or transmission of infectious diseases between humans, non-human vertebrates, human to non-human vertebrates, non-human vertebrates to humans, etc.

The composition of this invention may prevent, treat, complement traditional treatments, or cure a variety of microbial infections in all vertebrates such as, but not limited to, farm animals, domesticated animals, sports animals, zoo animals, pets, laboratory animals, wild animals, etc., or any combination thereof. The examples of the farm animals are but are not limited to, pig, chicken, cattle, etc. Domesticated animals, such as are but not limited to, goat, bull, buffalo, bezoar, sheep, etc. Sports animals, for example, but not limited to, horse, fox, dog, etc. Examples of zoo animals are but are not limited to, zebra, rhino, alligator, snake, lizard, frog, bird, fish, etc. Pets may include but are not limited to, dog, cat, horse, etc. Examples of laboratory animals are but are not limited to rats, frogs, mice, etc. Examples of wild animals are but are not limited to, elephant, lion, cobra, etc. These animals may be of any age, including eggs, and there may be an overlap between the aforementioned animal groups. The eggs may benefit directly by spraying the composition onto them or indirectly from their mother being exposed to the composition.

Another embodiment of the invention may provide inhaled composition from the feed or another so that the lung infections may be treated, complement traditional treatments, cured, prevented, or any combination thereof. Administered composition via any route may enter the systemic circulating system, and hence, prevent, treat, complement traditional treatments, or cure or any combination thereof any other organ infection.

In an embodiment of this invention, the composition may be non-toxic and or without side for the user, wherein users may be humans or non-human vertebrates exposed to the composition.

An embodiment of this invention may relate to preventing the spread of microbial from humans to humans, from humans to non-human vertebrates, from non-human vertebrates to humans, from non-human vertebrates to other non-human vertebrates including inter- and intra-species.

There is a huge market need for such a composition because this invention may prevent future pandemics and epidemics. Since this composition may prevent transmission from one non-human vertebrate to another non-human vertebrate, from non-human vertebrate to human, from human to a non-human vertebrate, and from human to human, this composition may prevent future of pandemic and epidemic. The non-human vertebrate to the non-human transmission may be between two or more species or maybe within the same species.

An embodiment of the invention may enhance the absorption of nutrients by improving gut health so that it may improve the overall health of all vertebrates or users. The composition may improve or enhance the growth of young vertebrates, e.g., chicks, etc.

An embodiment of the composition may be anti-bacterial, e.g., retard and/or kill all Salmonella species, E. coli, Campylobacter jejuni, etc., or any combination thereof.

An embodiment of the composition may be anti-viral, e.g., retard and/or kill Newcastle, avian influenza, Ebola, ASF virus, etc., or any combination thereof.

An embodiment of the composition may be anti-yeast, e.g., retard and or kill Candida albicans, etc.

An embodiment of the composition may be anti-fungi, e.g., retard and/or kill Aspergillus fumigatus, etc.

An embodiment of the composition may be anti-parasites, e.g., retards and/or kill all species of coccidia, etc.

An embodiment of the composition may be anti-rickettsia, e.g., retard and/or kill rickettsia, etc.

An embodiment of the composition may be anti-mites, e.g., retard and/or kill mites, etc.

An embodiment of the composition may be anti-micro-algae, e.g., retard and/or kill micro-algae, etc.

An embodiment of the composition may be anti-Salmonella species, such as S. typhimurium, S. heidelberg, S. hadar, S. seftenberg, e.g., retard and/or kill these species.

An embodiment of the composition may be anti-Clostridium perfringens, e.g., retard and/or kill Clostridium perfringens, etc

An embodiment of the composition may be anti-MRSA (methicillin resistant Staphylococcus aureus), e.g., retard and/or kill methicillin resistant Staphylococcus aureus, etc

An embodiment of this invention may be manufactured in any form such as, but not limited to, mixture, amalgamate, solutions, patches, pellets, pills, oral-deliverables, injections, anal-deliverables, inhalable, emulsion, hydrogel, hydrogel admixture, pressurize or non-pressurized spray, seed cover, seed coat, paste, capsule, syringe filling, mash, unmixed, mixed, blendable, unblendable, miscible, immiscible, or any combination thereof.

Example: The presently disclosed subject matter is better understood from reading the following example and associated experimental data and results along with the description of non-limiting embodiments mentioned herein. The example illustrated herein uses a composition A and composition B, and a methodology for each experiment are described in each figure. The composition had a final total of volume of 40 gallons.

COMPOSITION A

| Ingredient | Qty/lbs | Range (% wt/vol) |
|---|---|---|
| Coconut Oil | 61 | 18.3%-25.9% |
| Chia Flour | 50.3 | 12.3%-19.9% |
| Krill Oil | 50.2 | 17.4%-30.2% |
| Oregano Oil | 50.2 | 13.3%-19.8% |
| Fumed Silica | 24 | 2.4%-8.9% |
| Coriander Powder | 10.1 | 1.5%-7.8% |
| Cranberry Seed Oil | 8.5 | 1.2%-7.4% |
| Ginger Powder | 2.1 | 0.3%-1.8% |
| Black Cumin Oil | 1.9 | 0.3%-1.7% |
| Carob Powder | 2 | 0.2%-1.6% |
| Fenugreek Powder | 1.3 | 0.1%-1.2% |
| Cardamom Powder | 1.1 | 0.1%-1.1% |
| Agaricus Mushroom | 0.9 | 0.1%-1.0% |
| Powder | | |

| | COMPOSITION B |
|---|---|
| Ingredient | Range (% wt/wt) |
| Oregano Oil | 11.0 |
| Organic Certified Coconut oil | 24.5 |
| Elantria DHA 400 Algae | 18.8 |
| MCT Oil | 25.0 |
| Cranberry Seed oil | 3.5 |
| Coriander oil | 3. |
| Ginger oil | 1.0% |
| Black Cumin Oil | 1.0 |
| Carob oil | 1.0 |
| Fenugreek oil | 0.4 |
| Agaricus oil | 0.5% |
| Cinnamon oil | 0.8 |
| Lemon Oil | 9.5 |

| | COMPOSITION C |
|---|---|
| Ingredient | Range (% wt/wt) |
| Oregano Oil | 23.0% |
| Organic Certified Coconut oil | 20.8% |
| Elantria DHA 400 Algae | 15.0% |
| MCT Oil | 21.0% |
| Cranberry Seed oil | 3.7% |
| Coriander oil | 2.6% |
| Ginger oil | 1.4 |
| Black Cumin Oil | 0.5% |
| Carob oil | 0.5% |
| Fenugreek oil | 0.5% |
| Agaricus oil | 0.6% |
| Cinnamon oil | 1.0% |
| Lemon Oil | 10.4% |

Prepared microbial overnight cultures and adjusted the culture Optical Density (OD) to 1 (approximate 10⁸ CFU/ml), inoculated 10 μl (microliters) of the OD₆₀₀ of 1 culture dilutions into 10 ml of LB (approximately 10⁵ CFU/ml). Either Composition A, B, or C were added to each of the experimental cultures (100× final dilution) but not to the control experiments. The inoculated cultures were incubated in a shaking incubator at 37° C. and 200 rpm for 3 hours. The cultures were serially diluted and plated on L-Agar and then incubated at 37° C. for overnight. On the next day, the numbers of colonies on each plate were counted.

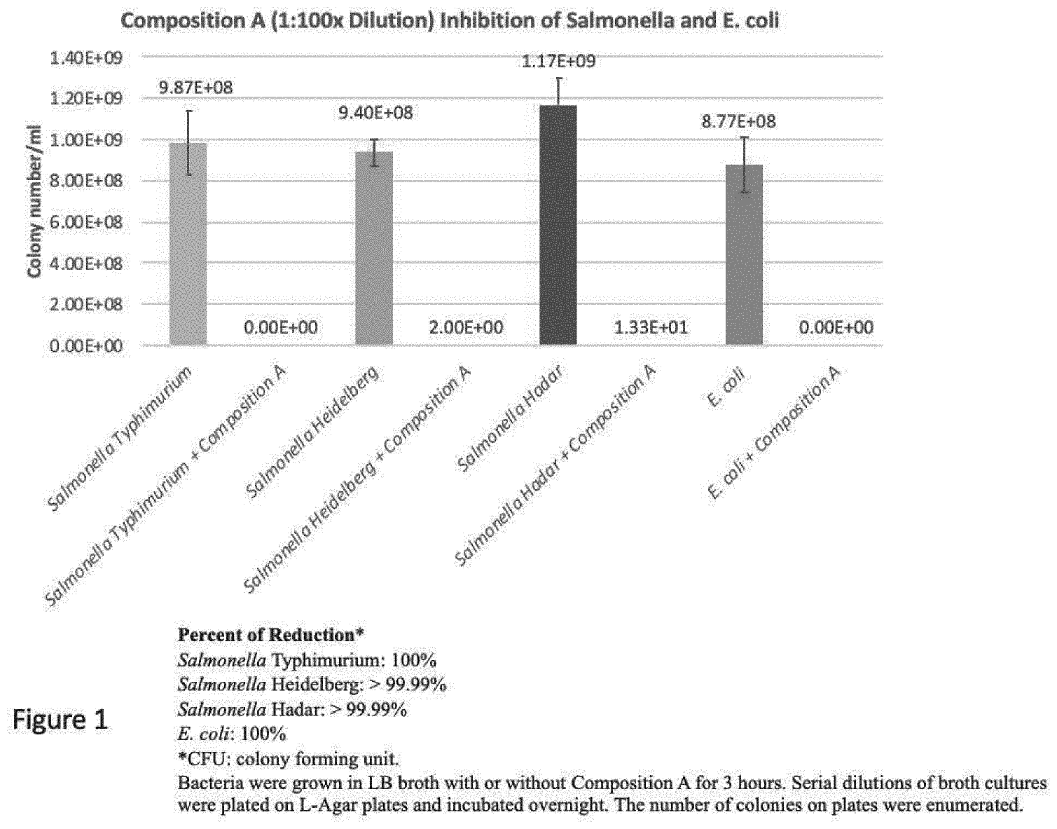

FIG. 1: Composition A/B/C (1:100× dilution) inhibition of Salmonella and E. coli: About 100% reduction for both Salmonella species and E. Coli, as shown in FIG. 1. An average of at least three different experiments.

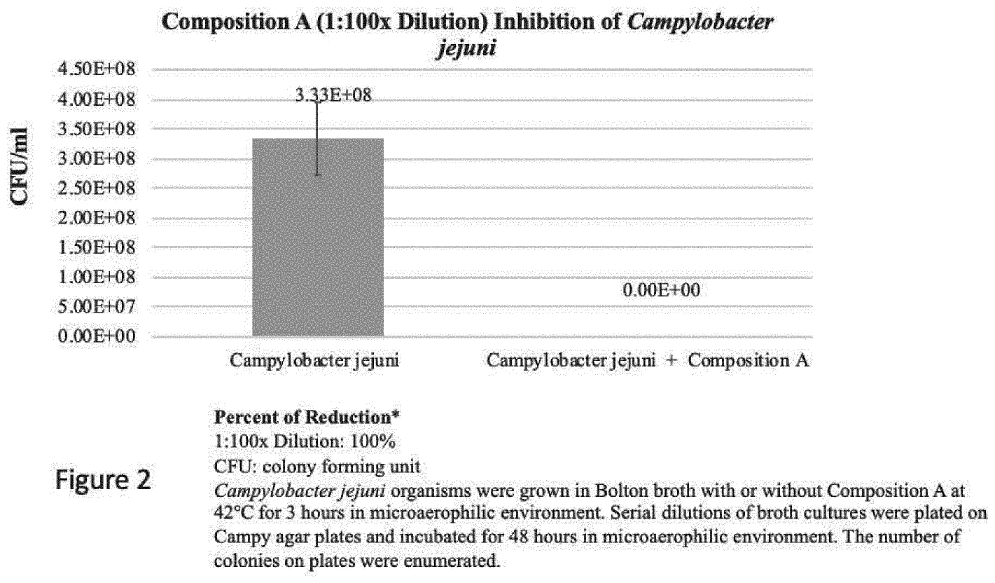

FIG. 2: Composition A/B/C (1:100× Dilution) Inhibition of Campylobacter jejuni. 100% reduction of Campylobacter jejuni, as shown in FIG. 2. An average of at least three different experiments

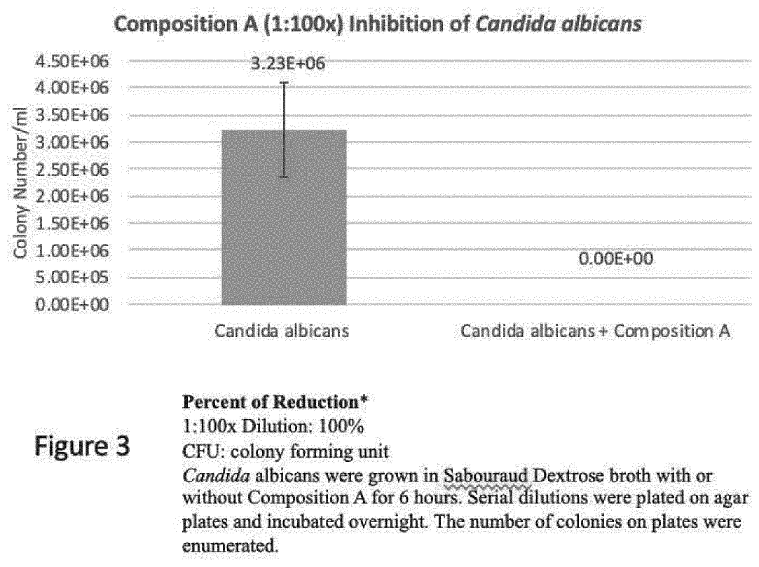

FIG. 3. Composition A/B/C (1:100× Dilution) Inhibition of Candida albicans. 100% reduction of Candida albicans, as shown in FIG. 3. An average of at least three different experiments

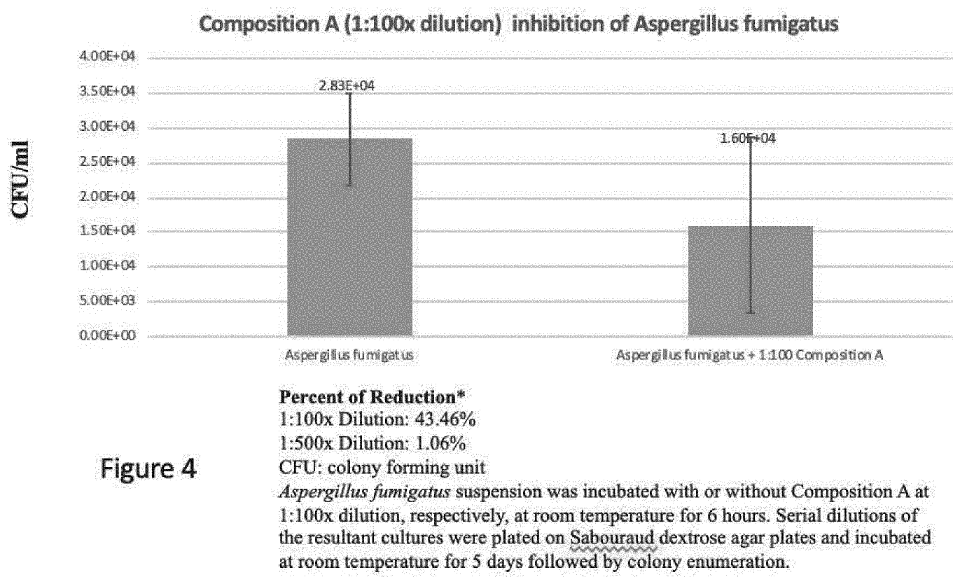

FIG. 4. Composition A/B/C Inhibition of Aspergillus fumigatus. Composition A/B at a dilution of 100× reduced the Aspergillus fumigatus by about 44%, an average of at least three different experiments, as shown in FIG. 4.

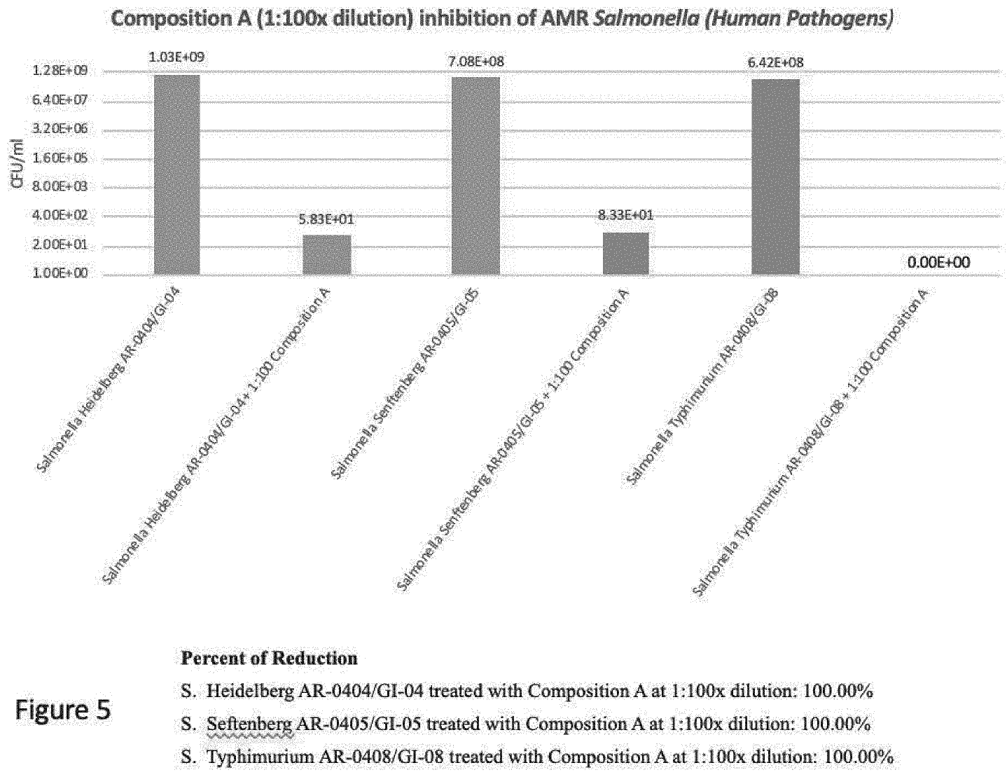

FIG. 5. Composition A/B/C Inhibition of (Anti-Microbial Resistance) AMR Salmonella, Even Salmonella species resistant to known anti-microbial exhibited significant inhibition of about 100%, an average of at least three different experiments. These human infecting AMR Salmonella cause serious diseases in humans and are highly resistant to several antibiotics. The results presented in this figure are unexpected and extremely beneficial in the treatment of drug-resistant bacterial infections in humans.

All three compositions showed similar results; however, all data not shown compactness.

Microbial s were tested in two independent laboratories, from the UMC-VMSL in Columbia, Mo., and microbe InoTech of St Louis. The microbes were provided by the two laboratories above and known potentially lethal antibiotic-resistant Salmonella species were provided by the CDC/DDID, Atlanta. Clostridium perfringens MRSA (methicillin resistant Staphylococcus aureus), Salmonella species. (S. typhimurim, S. heidelberg, S. senftenberg, S. hadar) (repeated with AMR (anti-microbial resistant) species of Salmonella to include S. typhimurium, S. heidelberg, these last 3 are from CDC and are different variants of the prior Salmonella. Chemicals were purchased from Fisher-Scientific, and herbs were purchased from numerous commercial sources such as American Botanicals.

The results presented in all the above compositions A, B, and C are unexpected, unpredictable, and of significant benefit for all vertebrates. Several compositions were tested, but only a few (including the three presented herein) showed compositions provided the surprisingly unexpected results given herein. Quite unexpectedly, we observed that the composition inhibited all bacterial species, yeast, and fungi from surviving at 99.9-100% incident with an average of 10⁶-10⁸ log change, heralded by numerous industry professionals as ‘quite impressive’ and ‘exciting.’ The composition of this invention with the surprising and unpredictable anti-microbial activity contains non-toxic, side-effect free, all natural, essential plant oils, have never before been commercially available in the measured formulation of this invention. The compilation of the ingredients of the composition of this invention is useful in both preventative health maintenance, and for active clinical illness in any vertebrate species, including humans. Thus, this invention is novel and nonobvious.

An embodiment of the composition has anti-microbial activity against drug resistance microbes such as, but not limited to, AMR Salmonella species described herein and others.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims and may include other examples that occur to those skilled in the art. Such other examples are intended to be within the scope of the claims if the examples have structural elements or steps that do not differ from the literal language of the claims, or if the examples include equivalent structural elements or steps with insubstantial differences from the literal language of the claims.

What is claimed is:

1. An anti-microbial composition comprising coconut/MCT oil, Elantria DHA 400 algae, cranberry seed, black cumin, oregano, carob, and Agaricus mushroom; wherein the composition is side-effect-free in any vertebrate; and wherein the composition is non-toxic to any vertebrates.

2. The composition of claim 1, further comprises at least one or more selected from chia, krill, micro-algae, cinnamon, lemon, coriander, ginger, peppermint, fenugreek, moringa, insect protein, cardamom, or any combination thereof.

3. The composition of any of claim 1, wherein krill comprises from 5% to 40% by weight/volume, wherein micro-algae comprises from 5% to 40% weight/volume, wherein cranberry seed comprises from 0.1% to 10% by weight/volume, wherein chia comprises from 5% to 30% by weight/volume, wherein black cumin comprises from 0.05% to 5% by weight/volume, wherein carob comprises from 0.1% to 5% by weight/volume, and wherein cardamom comprises from 0.01% to 5% by weight/volume or any combination thereof.

4. The composition of claim 1, wherein oregano oil 11-24%, coconut oil 19-24.5% wt/wt, wherein elantria DHA 400 algae comprise 15-20% wt/wt, wherein MCT oil comprises 21-26.5% wt/wt, wherein cranberry see oil comprises 2.0-4.5% wt/wt, wherein coriander oil comprises 2.-4% wt/wt, wherein ginger oil comprises 0.4-1.5% wt/wt, wherein black cumin oil comprises 0.2-1.5% wt/wt, wherein carob oil comprise 0.2-1.5% wt/wt, wherein fenugreek oil comprises 0.2-1.5% wt/wt, wherein Agaricus oil comprises 0.2-1.5% wt/wt, wherein cinnamon oil comprise 0.2-1.5% wt/wt, wherein lemon oil comprise 8.4-10.5% wt/wt, or any combination thereof.

5. The composition of claim 1, wherein it can be used at a dilution of 1:100, e.g., 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95.

6. The composition of claim 1, further comprising at least one or more oil dilutants selected from coconut oil, peanut oil, soybean oil, corn oil, canola oil, cottonseed oil, olive oil, palm oil, rapeseed oil, safflower oil, sesame oil, pumpkin oil, almond oil, grape seed oil, avocado oil, flax-seed oil, sunflower, or any combination thereof; wherein the oil is extracted mechanically, chemically, or a combination of both.

7. The composition of claim 1, further comprises at least one or more thickener selected from fumed silica, natural gum, starch, gelatin, agar, arrowroot, pectin, psyllium, sago, dextrin, cellulose, and tapioca, tapioca starch, Xanthum gum, Kuzu/kudzu, Polyethylene glycol (PEG), waxes, or any combination thereof.

8. The composition of claim 1, further comprising at least one or more flavors selected from mint, peppermint, spearmint, menthol, basil, clove, cilantro, fennel, eucalyptus, garlic, lavender, lemon, lemon-grass, citrus, orange, rose, sage, lemon oil, etc., or any combination thereof.

9. The composition of claim 1, wherein the composition is in a at least one or more physical forms of an emulsion, oil, oil mixture, hydrogel, hydrogel admixture, solutions, patches, pellets, pills, oral-deliverables, injections, anal-deliverables, inhalable, spray, top dressing, top coating, seed cover, seed coat, paste, capsule, syringe filling, unmixed, unblendable, blended, miscible, immiscible, mash covering, commercial food covering, commercial food coating, prepared food additive, prepared food covering, prepared food coating, or any combination thereof.

10. The composition of claim 1, wherein a dose is single or multiple doses e.g., 1, 2, 3, 4, 5, 6, 7, 8, 9, 10, 11, 12, 15, 16, 17, 18, 19, 20, 21, 22, 23, 24, 25, 26, 27, 28, 29, 30-35, 35-40, 40-45, 45-50 dose and any combination thereof.

11. The composition of claim 1, wherein the composition can be administered for minutes, hours, days, weeks, months, or years, e.g., 1, 2, 3, 4, 5, 6, 7, 8, 9, 10, 11, 12, 13, 14, 15, 16, 17, 18, 19, 20-25, 25-30, 30-35, 35-40, 40-45, 45-50, 50-55, 55-60 days, or any combination thereof, 1, 2, 3, 4, 5, 6, 7, 8, 9, 10, 11, 12, 13, 14, 15, 16, 17, 18, 19, 20, 21, 22, 23 weeks, or any combination thereof, 1, 2, 3, 4, 5, 6, 7, 8, 9, 10, 11, 12, 13, 14, 15, 16, 17, 18, 19, 20, 21, 22, 23 months, or any combination thereof, 1, 2, 3, 4, 5 years, or any combination thereof.

12. The composition of claim 1, wherein the composition is administered indirectly as a food additive, supplement, or a combination of both for vertebrates, wherein the vertebrates are humans, pets, farm animals, zoo animals, laboratory animals, wild animals, and domesticated animals.

13. The composition of claim 1, wherein the composition dose(s) are directly administered to vertebrates, wherein the vertebrates are humans, pets, farm animals, zoo animals, laboratory animals, wild animals, and domesticated animals.

14. The composition of claim 1, wherein the anti-microbial activity is against single cellular or multicellular organisms, e.g., bacteria, viruses, yeast, fungi, parasites, mites, or any combination thereof.

15. The composition of claim 1, wherein anti-microbial activity is against Salmonella species, S. typhimurium, S. heidelberg, S. hadar, S. seftenberg, E. coli, Campylobacter jejuni, Clostridium perfringens, MRSA (methicillin resistant Staphylococcus aureus), Newcastle, avian influenza, Ebola, ASF virus, Candida albicans, Aspergillus fumigantus, species of coccidia, rickettsia, or any combination thereof.

16. The composition of claim 1, wherein the composition is used alone or in combination with other prescribed or non-prescribed other anti-microbials for enhanced results.

17. The composition of claim 1, wherein the composition provides a nutritious value to the user vertebrate; and wherein the composition anti-microbial activity is drug resistant.

18. The composition of claim 1, wherein the composition enhances the growth and health of chicks, turkey poults, and other young vertebrates users; and wherein the composition decreases morbidity and mortality in any vertebrate user.

19. A method of prevention, treating, complementing traditional treatments, or cure of microbial infections from a non-human vertebrate to human, and from human to a non-human vertebrate comprising:
i) indirectly dosing by adding the anti-microbial composition does to the food or direct dosing with a composition comprising of two or more selected from krill, cranberry seed, chia, black cumin, carob, and cardamom; wherein the mixture is an emulsion, hydrogel, hydrogel admixture, solutions, patches, pellets, pills, oral-deliverables, injections, anal-deliverables, inhalable, spray, top dressing, top coating, seed cover, seed coat, paste, capsule, syringe filling, unmixed, unblendable, blended, miscible, immiscible, mash covering, commercial food covering, commercial food coating, prepared food additive, prepared food covering, or prepared food coating,
ii) dosing the said composition into single or multiple doses;
iii) administering the said composition in the said doses;
iv) repeating the steps (i) to (iii).

20. A method of preventing the microbe transmission from a non-human vertebrate to human comprising:
i) indirectly dosing by adding the anti-microbial composition does to the food or direct dosing with a composition comprising of two or more selected from krill, cranberry seed, chia, black cumin, carob, and cardamom; wherein the mixture is an emulsion, hydrogel, hydrogel admixture, solutions, patches, pellets, pills, oral-deliverables, injections, anal-deliverables, inhalable, spray, top dressing, top coating, seed cover, seed coat, paste, capsule, syringe filling, unmixed, unblendable, blended, miscible, immiscible, mash covering, commercial food covering, commercial food coating, prepared food additive, prepared food covering, or prepared food coating,
ii) dosing the said composition into single or multiple doses;
iii) administering the said composition in the said doses;
iv) repeating the steps (i) to (iii);
wherein the vertebrate is a pet, domesticated animal, zoo animal, laboratory animal, wild animal, farm animal, or any combination thereof.

21. A method of claim 20, wherein the transmission of microbial from a human to other humans is prevented.
